(19) United States
(12) Patent Application Publication
(10) Pub. No.: US 2014/0207049 A1
(43) Pub. Date: Jul. 24, 2014

(54) ENERGY CHIP STRUCTURE

(71) Applicant: Overtake Technology Co., Ltd., New Taipei City (TW)

(72) Inventor: Huang Tien-Yu, New Taipei City (TW)

(73) Assignee: Overtake Technology Co., Ltd., New Taipei City (TW)

(21) Appl. No.: 14/162,457

(22) Filed: Jan. 23, 2014

(30) Foreign Application Priority Data
Jan. 23, 2013 (TW) 102201466

(51) Int. Cl. A61N 5/06; A61L 31/16

(52) U.S. Cl. CPC A61N 5/0613; A61L 31/16
USPC 604/20; 607/100

(57) ABSTRACT

The present invention provides an energy chip structure comprising: a base, an energy chip and a protective layer; wherein the base includes an inner space, the energy chip is arranged inside the inner space and for emitting a stabilizing electric wave, and the protective layer is attached to a top of the energy chip and covered the inner space via a packaging and storage method. Accordingly, the energy chip structure of the present invention is able to provide energy beneficial to the health of human bodies.

### FIELD OF THE INVENTION

The present invention relates to a structure of an energy chip, and particular to an energy chip structure capable of emitting a stabilizing electric wave beneficial to the health of human bodies.

### BACKGROUND OF THE INVENTION

With the rapid technology development nowadays, various types of health products are developed and available in the market, including titanium necklaces, germanium rings, germanium and titanium rings, far-infrared decorative accessories, and other relevant ore materials or energy products. Disregarding the medical effects or treatments, such energy related products are aimed to facilitate the regulation of the physiological functions or the body or to improve the body fitness and health.

With respect to the aforementioned health products related to the ore materials, they are able to release an energy to indirectly improve the fitness and health of the body. In fact, for products containing ore materials, the ore must be grinded into a powder form to be blended into decorative accessories. In addition, since most ore materials contain numerous elements including both positive ions and negative ions therein accordance with the research finding in the medical field that the positive ions, known as the “fatigue-related ions”, tend to have negative impacts on the biological body health while the negative ions, known as the “vitality-ion” can have positive influences on the health of the body, such products using ore materials directly therein cannot be assured of the content of the positive energy related to the aforementioned vitality ions. Furthermore, there are more energy products of the aforementioned types need to be worn for a significant period of time in order to have the desired effects to take place; consequently, consumers are likely to have negative impressions on such products as well as low trusts on the use of such products.

In view of the above and the numerous drawbacks associated with the prior arts, the present invention provides an energy chip containing a structure capable of collaborating the content of the negative ions and the far-infrared related technology along with the application of the theory of molecular resonance. By that the user wearing or using a product of the present invention can be benefited from the energy chip of the present invention in terms of the health effects generated thereby to provide enhanced physical conditions and greater health as well as improved body fitness to the user.

### SUMMARY OF THE INVENTION

In view of the aforementioned drawbacks associated with the prior arts, the present invention provides an energy chip structure.

An energy chip structure of the present invention comprises a base having an inner space formed therein, an energy chip arranged inside the inner space and used for emitting a stabilizing electric wave; a protective layer attached to the top of the energy chip in order to cover the inner space via a packaging and storage method. Accordingly, the energy chip structure of the present invention is able to provide positive influences on the health of human bodies.

In comparison to the prior arts, the energy chip structure of the present invention has at least the following merits:
(1) The energy chip structure is capable of emitting a stabilizing electric wave including far-infrared to enhance the blood circulation and increase the activation of the body cells as well as to stimulate the production of enzymes.
(2) The energy chip structure comprises contents of negative ion silicon to assist the activation of the cells, the purification of the blood and the increase of body resistance and strength against viruses as well as the adjustment of autonomic nervous system.
(3) The energy chip structure can be embedded with a certain wavelength frequency which is capable of creating a resonance with the water molecules in the body in order to stimulate the metabolism of the body.
(4) The energy chip structure is capable of promoting the generation of hemoglobin increasing the blood flow, flow speed and the oxygen content in the blood.
(5) The energy chip structure is adaptable to be configured in a decorative accessory; the energy can be released to the human body of the user wearing such accessory in order to enhance the health effects on the body.
(6) The energy chip structure is configured to be worn by the user in order to activate the body cells in a relatively short period time while allowing the user to test his or her benefited body improvements including such as body endurance, body impacts and resistance to greater physical forces as well as body flexibility.
(7) The energy chip structure has the properties of being non-invasive, non-toxic, antimicrobial and environment-friendly and for easy cleaning thereof.

### BRIEF DESCRIPTION OF THE DRAWINGS

FIG. 1 is an exploded view showing an energy chip structure of the present invention;

FIG. 2 is an illustration showing the energy chip of the energy chip structure of the present invention;

FIG. 3 is a perspective view showing an assembly of the energy chip structure of the present invention; and

FIG. 4 is a preferred embodiment of the energy chip structure of the present invention.

### DESCRIPTION OF EMBODIMENTS OF THE INVENTION

To understand the objectives, features and effects of the present invention, the following detailed description of the embodiment of the present invention is provided along with the accompanied drawings to further describe the present invention in greater detail as follows.

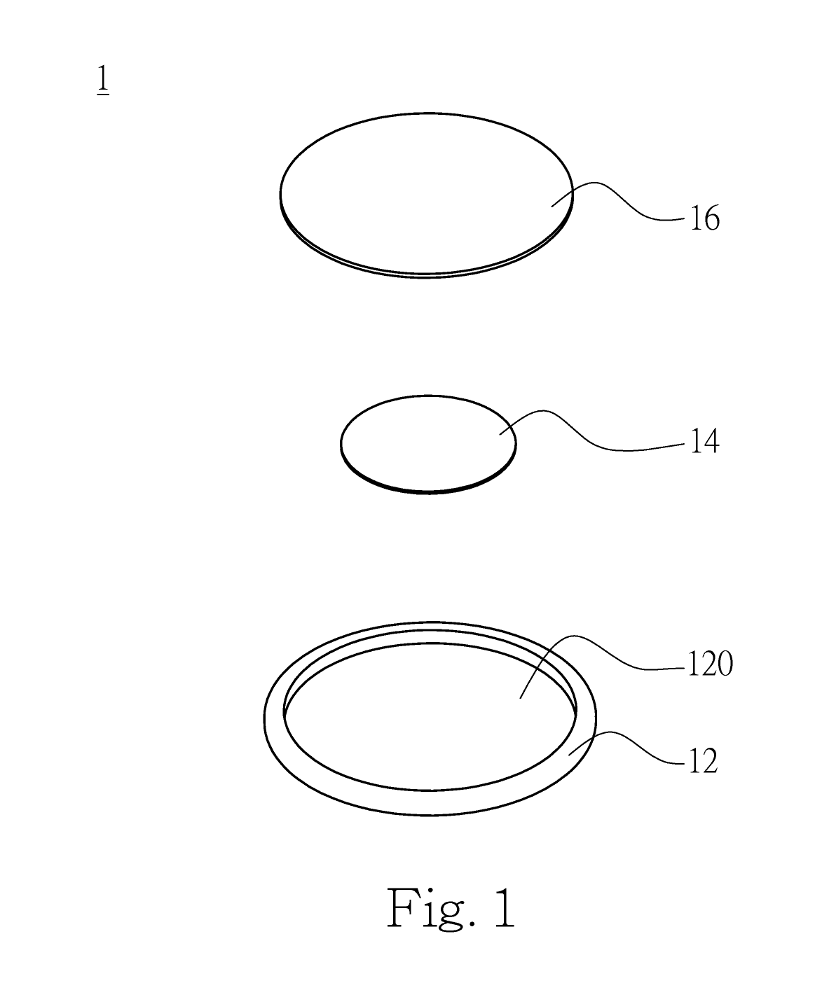

Please refer to FIG. 1 showing an exploded view of an energy chip structure of the present invention. The energy chip of the present invention comprises: a base 12, an energy chip 14 and a protective layer 16.

The base 12 includes an inner space 120. The energy chip 14 is arranged inside the inner space 120 and emitted a stabilizing electric wave. The protective layer 16 is attached to the top of the energy chip 14 and covered onto the inner space 120 via a packaging and storage method. According to a preferred embodiment of the present invention, the base 12, the energy chip 14 and the protective layer 16 are of an outer shape selected from any one of a circular shape, a diamond shape, a prism shape, a heart shape, an oval shape and a polygonal shape respectively.

In addition, the base 12 and the protective layer 16 are preferably made of a material selected from any one of a silicon, a rubber and an environmental-friendly resin respectively. Furthermore, the material preferably comprises a content of negative ions and has the properties of non-invasive, non-toxic, antibacterial and environment-friendly as well as for easy cleaning thereof.

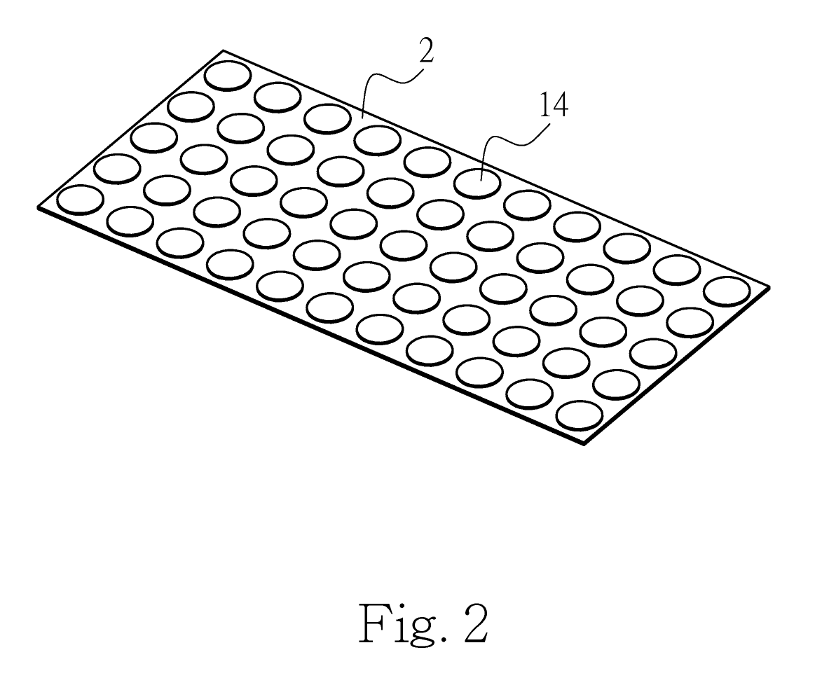

Please refer of FIG. 2 showing an illustration of the energy chip of the energy chip structure of the present invention. The plate 2 includes the plurality of energy chips 14 disposed thereon, and the plurality of energy chips 14 are of a predefined particular wavelength frequency, such that it is embedded into the energy chips 14 by an operating machine (not show in the figure); wherein the wavelength frequency includes far-infrared and stabilizing electric wave.

Furthermore, the energy of far-infrared emitted by the energy chip 14 are named as the “life light” by scientists in the field; also, according to theoretic physics, the energy of the far-infrared at a wavelength similar or identical to that of a human body can be easily and quickly absorbed by the human body once such far-infrared is shone toward the human body. Once the human body absorbs such energy of the far-infrared, the blood circulation of the body can be increased to promote the metabolism, such that wounded body cells can be healed and tissue cells can be activated along with the stimulation on the generation of enzymes.

In addition, with the energy of the far-infrared emitted by the energy chip 14, the stabilizing electric wave emitted therefrom is able to enter the human body with ease. Furthermore, the vibration of the body cells and molecules are able to merge the water molecules of smaller molecular groups into the cell membranes, in order to provide and supplement the water necessary for the body cells. Therefore, the nutrients required by the body cells can be effectively provided and the metabolism can be increased. Moreover, according to a test report conducted by the Industrial Technology Research Institute (ITRI) of Taiwan R.O.C., the energy chip 14 has an average emissivity of 0.839 of 4-14 μm at the temperature of 40° C.

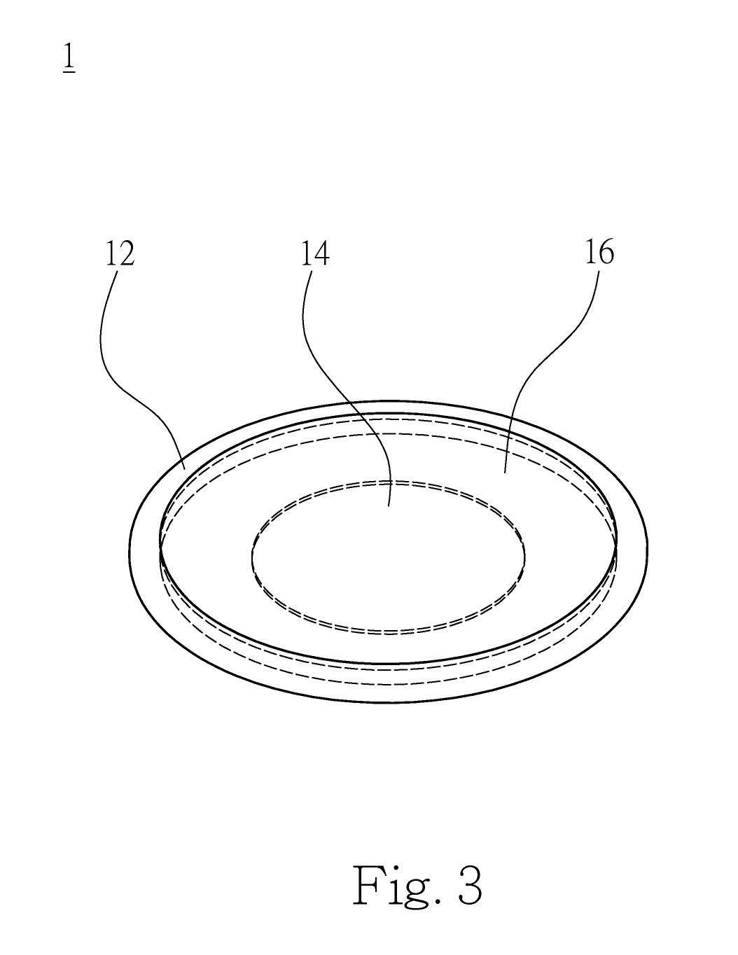

Please refer to FIG. 3 showing an illustration of an assembly of the energy chip structure of the present invention. The energy chip 14 is arranged in the inner space 120 of the base 12 and the protective layer 16 is then completely sealed to cover the energy chip 14 inside the inner space 120 via a packaging and storage method.

During the release process of the energy from the energy chip 14, the protective layer 16 is able to isolate the energy chip 14 from contacting with the ambient air such that useful time of the energy chip structure 1 the can be prolonged. In addition, the base 12 and the protective layer 16 both include contents of negative ions such that they can be helpful in assisting the vitalization of the cells, purification of the blood, resistance against viruses and adjustment of autonomic nervous system. Moreover, according to a test report conducted by SGS, the energy chip 14 has a content of negative ions of 897 ions/cubic centimeter at the temperature of 26° C. and humidity of 49% (RH).

Additionally, according to a test report conducted by Taiwan Textile Research Institute (TTRI), under the environment condition at the temperature of 20° C. and the humidity of 65% (RH), a difference between the blood flows with and without the use of the energy chip structure 1 is +17.2%, which means the blood flow is increased by 17.2% with the use of the energy chip structure 1; similarly, an increase in the flow speeds of the blood of +14.3% and an elevation of the skin temperature of +0.59° C. are found to be associated with the use of the energy chip structure 1. The above findings evidently indicate that the energy chip structure 1 is able to not only promote the generation of hemoglobin but also increase the oxygen content in the blood.

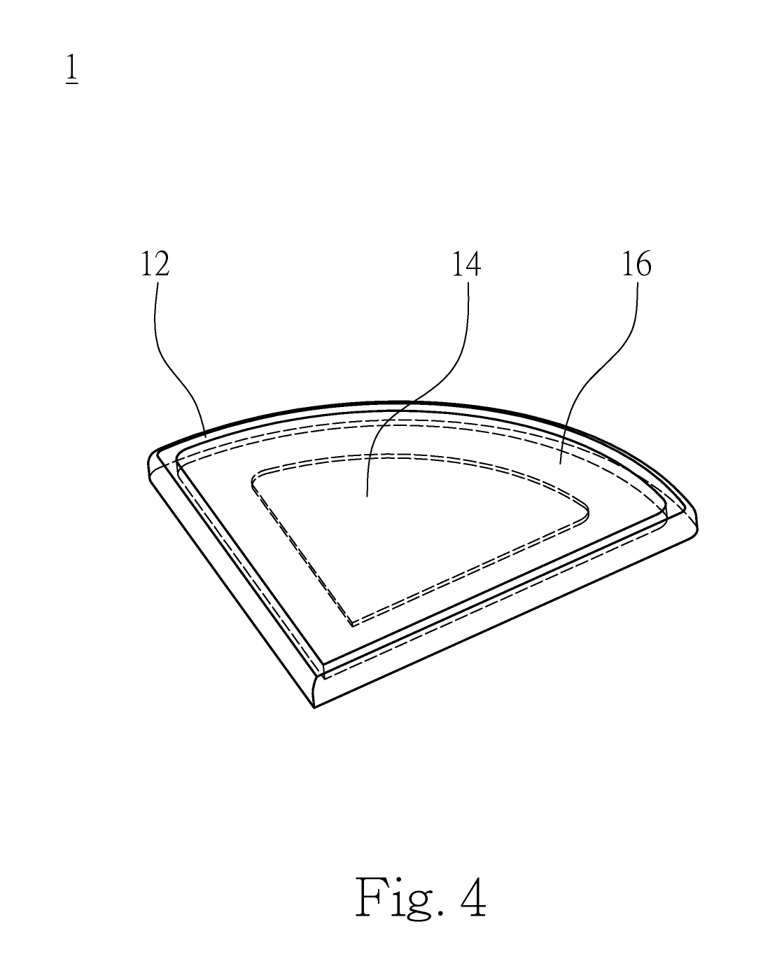

Please refer to FIG. 4 showing a preferred embodiment of the energy chip structure of the present invention. In this embodiment, it has an outer shape of a diamond such that the unique diamond shape of the energy chip 14 can be configured to match with a decorative accessory (not shown in the figure) with greater feasibility in addition to the enhancement in its appearance, beauty and uniqueness. Most importantly, it has been found that the energy being possessed by the energy chip 14 of a diamond shape is higher than the energy chips 14 of other shapes.

It shall be noted that the energy chip structure 1 can be configured to be worn by the user as a wrist band, necklace, ankle band or any other accessories including ones on the clothing of the user. In addition, the structure 1 of the energy chip is configured to be worn by any parts of the body or at any directions or angles to allow the user to test his or her own body improvements and various physically strengthened effects, inclusive of flexibility of muscles, elasticity strengthened, improved balance, increased power and endurance of the body, as well as the resistance to impacts that he or she may physically experience in addition to the benefit of the increase of the blood circulation.

The abovementioned embodiments are provided to illustrate the principles and exemplary methods of manufacturing or formation method of the present invention only. The scope of the present invention shall be defined by the claims recited hereafter, and any modifications or variations to the terms or wordings recited in the claims shall be considered as their relevant equivalence and are within the scope of the present invention. The scope of the present invention shall be determined by the content of the claims recited hereafter.

What is claimed is:

1. An energy chip structure comprising:
a base having an inner space formed therein;
an energy chip arranged inside the inner space and for emitting a stabilizing electric wave; and
a protective layer attached to a top of the energy chip and covered onto the inner space via a packaging and storage method.

2. The energy chip structure according to claim 1, wherein the base, the energy chip and the protective layer are of an outer shape selected from any one of a circular shape, diamond shape, a prism shape, a heart shape, an oval shape and a polygonal shape respectively.

3. The energy chip structure according to claim 1, wherein the base and the protective layer can be made of a material selected from any one of silicon, rubber and environment-friendly resin respectively.

4. The energy chip structure according to claim 3, wherein the material further comprising a content of negative ions.

5. The energy chip structure according to claim 3, wherein the material has properties of non-invasive, non-toxic, antibacterial and environment-friendly and for easy cleaning thereof.
